(19) United States
(12) Patent Application Publication
(10) Pub. No.: US 2011/0253563 A1
(43) Pub. Date: Oct. 20, 2011

(54) Currency sterilization apparatus

(76) Inventor: Edward Goldman, Framingham, MA (US)

(21) Appl. No.: 12/799,092

(22) Filed: Apr. 19, 2010

(51) Int. Cl. B65D 25/00

(52) U.S. Cl. 206/216

(57) ABSTRACT

A stand alone or retrofitted cash box that incorporates inner mounted ultra violet light sterilization and aerosol disinfectant release systems comprising automated actuation upon closing of the drawer, to reduce the amount of germs and bacteria found on currency both paper and coins at points of currency transaction. The retrofitted cash drawer component of the system comprises a perforated design to allow for greater surface exposure and currency desiccation quality.

### BACKGROUND OF THE INVENTION

1. Field of the Invention

The present invention relates to an apparatus for receipt, retaining, sterilization, disinfecting and disbursing of bank notes, more specifically to a currency handling device such as a cash box comprising an automated, internal sterilization and disinfecting processes.

2. Description of the Related Art

Proper receipt and handling of bank notes, particularly currency, has generally tended toward cash box type registers systems. Many systems have been developed for receipt, retaining and disbursal of currency, including such features as initiating a dispensing operation wherein bills sorted in money cases and a money receiving and disbursing box. Such systems may include automated pick-out devices for removal of the bills in the money cases where a quantity of the bills in the money cases is more than a predetermined value.

Further devices include cash transaction machines which include a sterilizing unit for sterilizing bills by heat, wherein the heating temperature of the sterilizing unit is maintained and detected by a sensor and maintained in a specified range. discloses a cash transaction machine performs receiving and/or paying transactions of bills by manipulation of a user, transfers bills between a receptacle where bills are paid in and out and a storage where bills are stored, and by using a sterilizing unit for heat-sterilizing bills being transferred at a specified temperature range and a specified time period.

Further disclosed are mechanisms for feeding a lengthwise oriented piece of paper dollar currency, or the like, in a C-shaped feed path and mounted on rollers about a centrally located ultraviolet source, which contributes to the sterilizing of the bill. Still further concurrent systems include a paper sterilization system comprising a chamber having one wall with an input slot and another wall with an output slot, a conveyor system comprising two co-acting belts in facing contact. While others disclose a method of sorting mail according to the invention includes the steps of receiving a plurality of first mail pieces of unknown origin in a first apparatus for bulk processing of the mail pieces, processing the mail pieces in the first apparatus by one of testing the mail pieces to determine if a potentially dangerous microorganism is present, sterilizing the mail pieces to destroy microorganisms on mail pieces, or both.

As is apparent, cleaning, washing, pressing of banknotes, particularly paper money is generally harmful and may reduce both the grade and the value of any note. Most assuredly, a washed or pressed note will lose its original sheen and its surface may become lifeless and dull. The existing defects within a note, such as folds and creases, may not necessarily be completely eliminated through washing or pressing and telltale marks can easily be detected under proper lighting conditions. Furthermore, carelessly washed notes may possess white streaks where the folds or creases existed. Thus, processing of a note which started out with a rating of Extremely Fine would certainly reduce the note at least one full grade.

Additionally, glue, tape, or pencil marks may often be successfully removed. While such removal will produce a clean surface, it will improve the overall appearance of the note without concealing any of its defects. Under such circumstances, the grade of the note may also be improved. Also, the words “pinholes”, “staple holes”, “trimmed”, “writing on face” and “tape marks” and the like should always be added to the description of a note as it is realized that certain countries routinely staple their notes together in groups before issue. In such cases, the description can include a comment such as “usual staple holes” or something similar in order to indicate to those would not otherwise know that this specific note cannot be found otherwise.

Thus, unfortunately, once currency is continually defiled, there exists no straightforward way to clean paper currency as conventional manners including utilization of soap and water, will not penetrate the surface of the bill.

### SUMMARY OF THE INVENTION

The instant device and system, as illustrated herein, is clearly not anticipated, rendered obvious, or even present in any of the prior art mechanisms, either alone or in any combination thereof. One object of the present apparatus is to provide a multi-configuration apparatus for receipt, retaining, sterilization, disinfecting and disbursing of bank notes, more specifically to a currency handling device such as a cash box comprising an automated, internal sterilization and disinfecting processes.

A further objective of the instant design is to introduce a stand alone, or retrofitted cash box that incorporates interior mounted ultra violet light sterilization and integrally disposed aerosol disinfectant release systems comprising automated actuation upon closing of the cash drawer, in order to reduce the amount of germs and bacteria found on currency both paper and coins at points of currency transaction (POS point of sale).

The present system relates to the sterilization of currency within a cash box, via the utilization of ultraviolet sterilization and disinfectant aerosol release. The system thus reduces the transmission of germs and bacteria at point of currency exchange during transactions. It is known that ultraviolet irradiation is successful in the elimination of germs and bacteria as applied in Laundromats (dry cleaning), ultraviolet sterilization for barbers tools, manicure equipment and dental tools. These applications have not been introduced to the cash register or ATM which is the main point of transfer for currency between people.

Broadly stated, the instant system provides a replacement for the standard cash box and cash drawer, which incorporates ultraviolet sterilization/irradiation and disinfectant expulsion within the cash drawer, once the drawer is closed and prior to re-opening of the draw. More specifically it is an object that provides-sterilization for use at a check out location for any retail location or place of currency exchange of which the currency can be exposed to sterilization in turn reducing the amount of germs and bacteria commonly found on currency and in turn reducing the transfer of germs between people.

Regarding usage with existent systems for currency storage and disbursal, such as cash registers, a new cash drawer will be supplied with each retro-fit for cash registers. This drawer may comprise a slightly greater height than drawers normally fitted in today's market-place. Located within the retrofitted drawer apparatus will be a metal frame that will be inserted secured to the inside of the drawer. This frame will act as the mounting points for either or both of an ultra violet introduction system and a disinfectant application system. The frame will act as wire management as well. Once the drawer is closed a pin actuator will turn on sterilization process and be controlled via a timer. The drawer kit/retro kit will incorporate a new cash drawer that is ventilated and transparent.

Further, as purpose of this project is to develop comprehensive currency sterilization at point of sale, point of sale is defined, but not limited to, cash registers, automated teller machines (ATM's), banks or other locations of currency transactions and transfers. Sterilization of currency prior to forwarding of said monies to another person or entity can help reduce transmission of known bacteria's, germs and other socially transferred disease. In effect we could lower potential spreading of common colds, viruses and socially common illnesses like H1N1.

Additionally, in one embodiment a new cash drawer comprised of high density poly propylene plastic and be ventilated on all areas similar to a strainer. This design would allow for airflow thus reducing any heat buildup and allowing for cross penetration. Additionally defined are embodiments of slim profile UV lamp system that could be mounted internally, thus enveloping the cash drawer with light rays. Additionally, as the currency on top of the drawer is the next to be distributed, a downward lamp would be of the utmost importance, especially for application similar to an ATM usage.

The other method of sterilization, which would employ a pressurized burst of common household disinfectant, which may also utilize booster fans for greater circulation. A simple pin actuator at the drawer closing point would act as start point of sterilization.

Phase two of product development would be the development, in conjunction with the largest manufacturer of cash registers and ATM's newly designed that would be specifically built two incorporate one or two of the technologies discussed above. Between the Severe Acute Respiratory Syndrome (SARS) and H1N1 outbreaks, a greater awareness of human hygiene and following of simple procedures which may save lives people throughout the world have become made us all more hygiene-conscious.

### BRIEF DESCRIPTION OF THE SEVERAL VIEWS OF THE DRAWINGS

As illustrated in FIG. 1, the instant system comprises a tube frame disposed to fit within the actual cash box of a register and further illustrates the disinfectant system which incorporates a series of nozzles disposed on the side walls of the cash box;

FIG. 2a illustrates the support frame disposed to retain the ultraviolet (UV) light panel array;

FIG. 2b illustrates the top panel which houses the Ultraviolet array or UV introduction system and the UV introduction system which comprises a series of bulb holders, mounted on a substantially horizontally disposed frame;

FIG. 3 illustrates the ultraviolet (UV) light panel array placed within the upper section of the support frame disposed to retain the ultraviolet (UV) light panel array.

FIG. 4 illustrates an internal view of one embodiment of the cash box mechanism utilizing an additional embodiment of UV lighting, a UV flex lighting fixture mounted on top of the cash box;

FIG. 5 illustrates the cash drawer mechanism, illustrating the paper currency containment chambers and the coinage containment chambers which double as and the divider walls; and,

FIG. 6 illustrates a bottom cash box UV lighting panel to be utilized with the embodiment exemplified in FIG. 5.

### DETAILED DESCRIPTION OF THE INVENTION

As illustrated in FIG. 1, instant invention comprises a tube frame 10, disposed to fit within an actual cash box 11 of an existing cash register system, and thus the cash box acts as an outer casing. In one embodiment, the dimensions may include a height of 3.5 and inches, a length of 15.75 inches and a width of 15.5 inches. This system would be enclosed within a cash box 11 of a height 3 and 15/16, width of 16 and ⅛ and a length of 16 and ⅜. The tube frame 10 may be disposed to move in and out of the cash box 11 along a pair of running tracks 19a-19b via wheels, bearings or any other slidably disposed mechanism as regularly used in the cash register or other such arts.

FIG. 1 additionally illustrates an embodiment of the disinfectant system wherein there exists a series of nozzles 12. At least three nozzles 12 should be utilized, one for each of the three side walls 13 of the cash box 11, wherein the nozzles are in fluid communication through a system of tubing 14 and disinfectant fluid is supplied through a supply reservoir apparatus 15 as known in the fluid arts. Additional nozzles 12 may be utilized for each wall and thus multiples of 3, including 6, 9 or 12 nozzles 12, may be utilized. A bladder system or a small pump system, as known in the fluid arts, may be utilized within the supply and reservoir apparatus 15 in order motivate the fluid through the tubing 14.

The disinfectant system will be triggered upon opening and closing of the drawer, by a pin actuator 18, or other similar mechanism, wherein upon depression of the pin actuator 18, an attached check valve mechanism, as known in the fluid arts, which is in fluid communication with the supply reservoir apparatus 15, is open to allow a metered flow of disinfectant, as the tube frame 10 moves in and out of the cash box 11 on the running tracks 19a-19b.

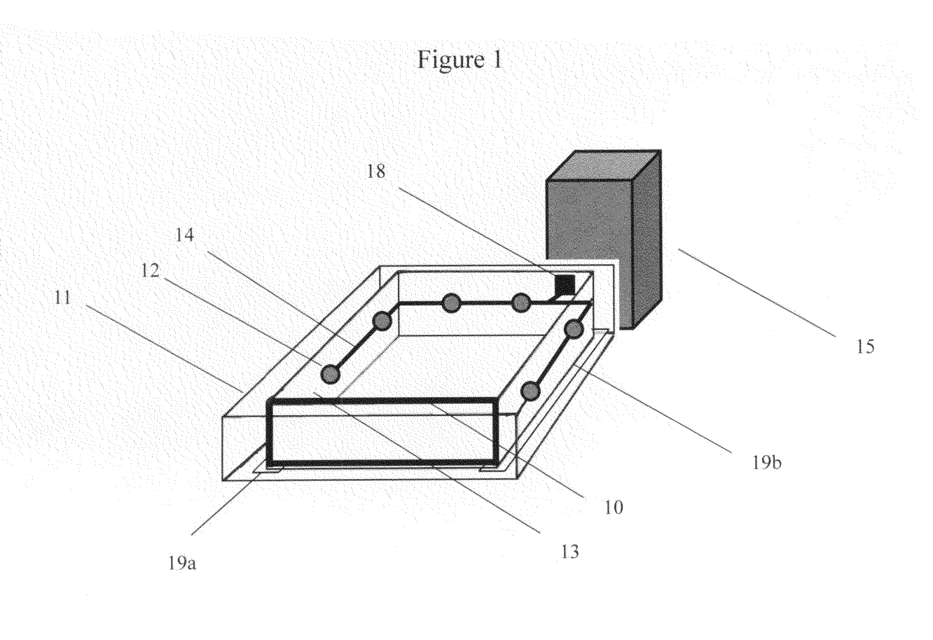

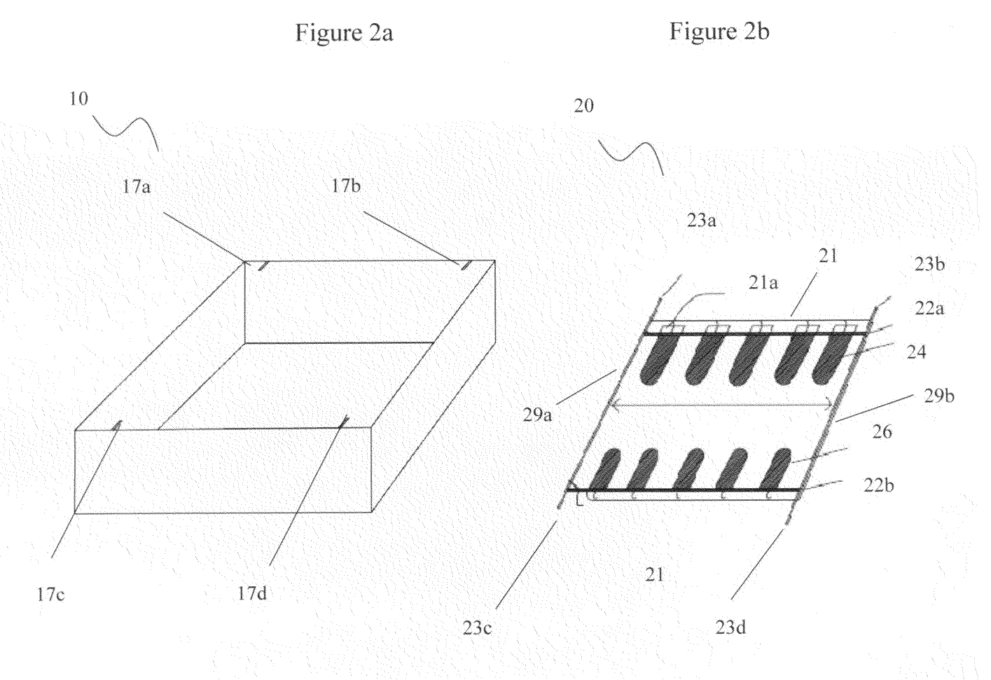

FIG. 2 further illustrates the tube frame 10 and specifically the receiving mechanisms 17a-17d, for attachment of the ultraviolet (UV) light panel 20. Two of these panels should be employed as a top panel 20a and a bottom panel 20b. FIG. 2 further illustrates the top panel or ultraviolet (UV) light panel 20 which comprises at least a bulb holder system 21 comprising at least one bulb holder 21a, mounted on at least two horizontal disposed tubes 22a and 22b or support beams, the horizontally disposed tubes mounted at opposing ends of the on either side and said panel 20. The horizontal support beams 22a and 22b maybe composed of any substantially non flammable material including steel, composite materials or any combination thereof. Additionally, the horizontally disposed tubes should be connected by at least two vertically disposed tubes 29a and 29b. Extending from the horizontal tubes 22a and 22b may be a series of mating protrusions 23a-23d, disposed for attachment to steel tube frame 10 to mate with the corresponding receiving mechanisms 17a-17d. The mating protrusions 23a-23d, and the corresponding receiving mechanisms 17a-17d, may be constructed as male to female mating systems.

Additionally, the ultraviolet (UV) light panel 20 may comprise a set of four inch (4″) uv tubes 24, in one embodiment, five (5) four inch (4″) UV tubes may be utilized. And, the ultraviolet (UV) light panel 20 may comprise a set of two inch (2″) UV tubes 26, in one embodiment five (5) four inch (2″) UV tubes may be utilized. The ultraviolet (UV) light panel 20 may also comprise a set of corresponding 4″ UV bulb adapters for the 4″ tubes and a set of 2″ UV bulb adapters for the 2″ UV tubes.

Further, the ultraviolet (UV) light panel 20 may comprise a power wire 27 and a corresponding lead to a power supply such as a one hundred and ten (110) volt source. The frame may be of a modular construction, or may also be composed of numerous pieces welded or formed together. The ultraviolet (UV) light panel 20 may be composed of any such material that can sufficiently meet the strength and heart tolerance requirements. Thus, numerous metals and composite material configurations, and combinations thereof, may be envisioned.

Much like the disinfectant system, the ultra violet panel may be designed in communication with a trip switch to power off and on with opening and closing of the drawer respectively, as know in the art. In that respect, both the ultraviolet and disinfectant systems may be electrically configured to be constantly running during register operation.

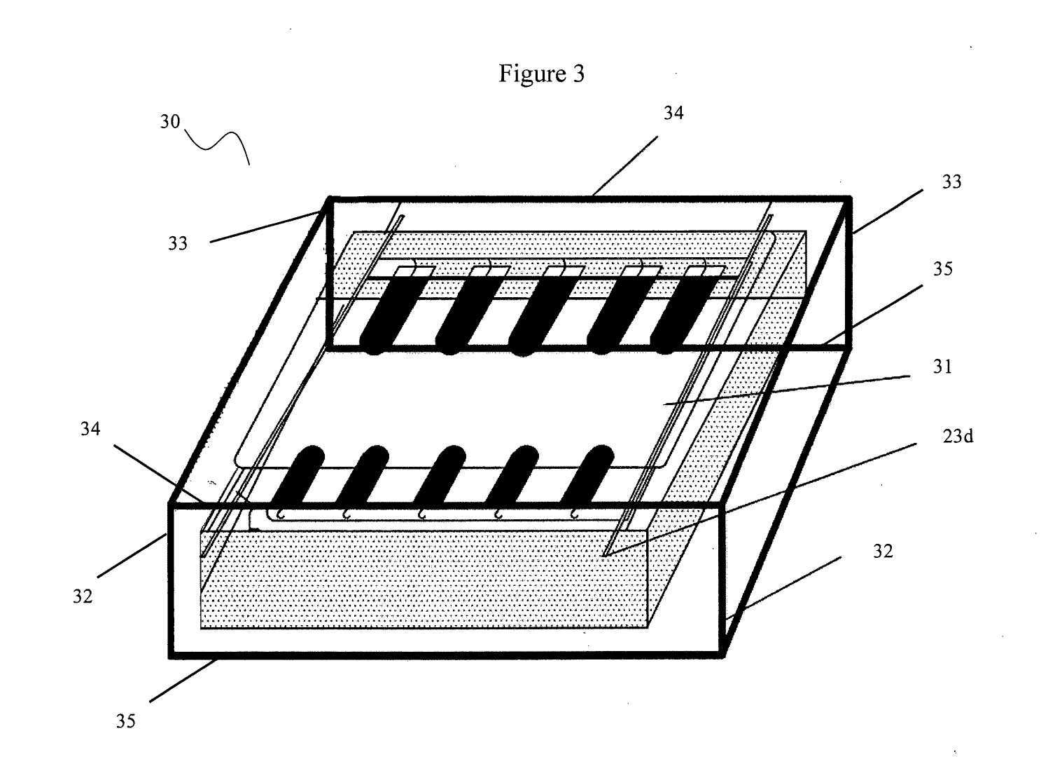

FIG. 3 illustrates the support frame 30 for the ultraviolet (UV) light panel array or system 20. The support frame 30 comprises a top panel 31, at least two frame upright backs (left and right) 32, at least two frame upright fronts (left and right) 33, a top bar 34, lower bar 35, the ultraviolet (UV) light panel 20 may be hung from both the upper and lower frame (as shown).

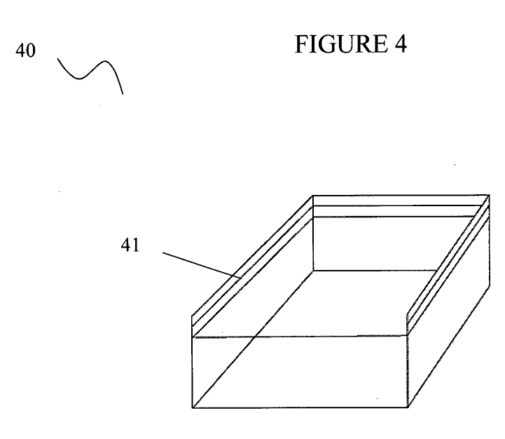

Additionally, FIG. 4 illustrates an internal view of one embodiment of the cash box mechanism utilizing an additional embodiment of UV lighting, a UV flex lighting fixture 41, mounted on top of the cash box.

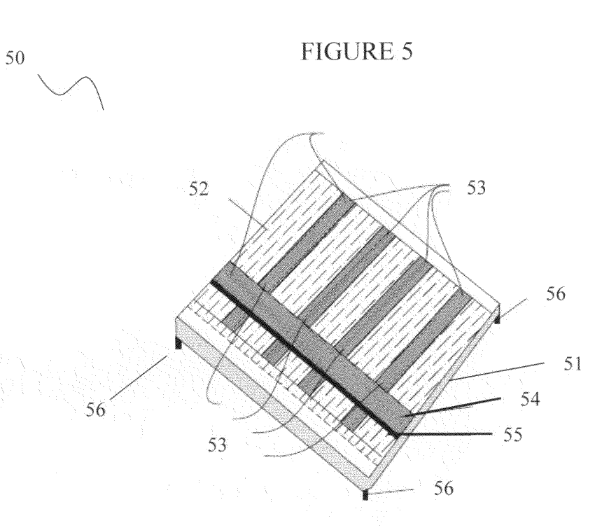

Further, FIG. 5 illustrates the cash drawer mechanism 50, wherein the cash drawer may be comprised of a molded design, which may be composed of transparent poly-propylene or any such polymeric material, and is designed to be ventilated to allow for increased exposure to airflow for the drying process. Thus, substantially all areas of the cash drawer mechanism 50, including the cash box side walls 51, may be perforated for increased exposure to disinfectant materials and for increased airflow. As an example and not meant to limit the material utilized, the cash drawer of the system may comprise materials such as clear poly propylene and/or perforated plastic to allow for greater surface exposure and currency desiccation.

Further illustrated, the paper currency containment chambers 52 may be physically defined be divider walls 53, and coinage may be stored within these divider walls 53. Again, both the containment chambers 52 and the divider walls 53 may also be perforated. The cash drawer mechanism 50 will be inserted underneath the UV light panel 20.

Additionally, it is envisioned to incorporate a front panel 54 for retaining currency in a proper position for cleaning, disinfection and latter currency disbursal. The front panel 54 may also be perforated and may also contain a UV strip 55. Included as well may be at least one footer mechanism 56, disposed at each lower side of the four corners of the cash drawer mechanism 50. The plurality of footer mechanisms 56 is designed to keep the cash drawer mechanism 50 from contacting the register floor and thus increase airflow. Thus, the plurality of footers 55 will afford the system the feature of bottom up sterilization.

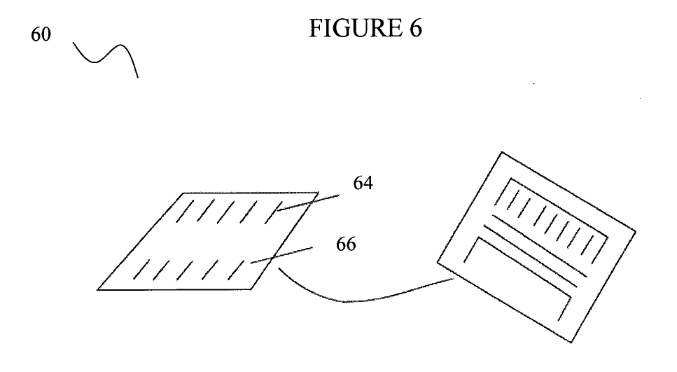

FIG. 6 illustrates a bottom cash box UV lighting panel 60 to be utilized with the embodiment exemplified in FIG. 5. The slim panel may be attached to interior of cash box to make a bridge like passage. Additionally, the ultraviolet (UV) light panel 60 may comprise a set of four inch (4″) UV tubes 64, in one embodiment, five (5) four inch (4″) UV tubes may be utilized. And, the ultraviolet (UV) light panel 60 may comprise a set of two inch (2″) UV tubes 66, in one embodiment five (5) four inch (2″) UV tubes may be utilized. The ultraviolet (UV) light panel 60 may also comprise a set of corresponding 4″ UV bulb adapters for the 4″ tubes and a set of 2″ UV bulb adapters for the 2″ UV tubes.

There has thus been outlined, rather broadly, the more important features of the portable electronics tester in order that the detailed description thereof that follows may be further understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty, which characterize the invention, are pointed out with particularity in the claims annexed to and forming a part of this disclosure. For a better understanding of the invention, its operating advantages and the specific objects attained by its uses, reference should be made to the accompanying drawings and descriptive matter in which there are illustrated preferred embodiments of the invention.

I claim:

1. A currency storage, purification and distribution system comprising:
a cash box system comprising:
an outer casing;
a ventilated cash drawer mechanism comprising at least two money case means for storing currency so that each money case means stores bills of different denomination from each other, money receiving and disbursing box means for receiving and storing bills irrespective of their denominations, said money receiving and disbursing box means being adapted for storing bills so that the bills may be readily added and removed by an operator;
an ultraviolet light administration system in communication with said cash box and disposed to provide a supply of ultraviolet light to a quantity of currency disposed within said cash drawer; and wherein said disinfectant system in communication with said cash box and disposed to provide a supply of disinfectant to a quantity of currency disposed within said cash drawer.

2. The currency storage, purification and distribution system wherein said ultraviolet light administration system and said disinfectant system are triggered upon closing of said cash box system.

3. The currency storage, purification and distribution system of claim 1 wherein said ultraviolet light administration system comprises an ultraviolet light panel comprising a set of four inch ultraviolet tubes.

4. The currency storage, purification and distribution system of claim 3 wherein said ultraviolet light panel further comprises comprising a set of five four inch ultraviolet tubes.

5. The currency storage, purification and distribution system of claim 1 wherein said ultraviolet light administration system comprises an ultraviolet light panel comprising a set of two inch ultraviolet tubes.

6. The currency storage, purification and distribution system of claim 5 wherein said ultraviolet light panel further comprises comprising a set of five two inch ultraviolet tubes.

7. The currency storage, purification and distribution system of claim 1 wherein said disinfectant system comprises at least three nozzles, wherein an individual of said at least three nozzles are located on one for each of the three side walls of said cash box.

8. The currency storage, purification and distribution system of claim 7 wherein said at least three nozzles are in fluid communication through a system of tubing and disinfectant fluid is supplied through a supply reservoir apparatus and a pump apparatus.

9. The currency storage, purification and distribution system of claim 8 wherein a quantity of said at least three nozzles is selected from the group consisting of multiples of six nozzles, nine nozzles and twelve nozzles.

10. The currency storage, purification and distribution system of claim 1 wherein the cash drawer mechanism comprises a plurality of paper currency containment chambers disposed in juxtaposition to a plurality of divider walls so as to be divided by each individual of said plurality of divider walls.

11. The currency storage, purification and distribution system of claim 10 wherein a quantity of coinage currency may be stored within said plurality divider walls.

12. The currency storage, purification and distribution system of claim 11 wherein said containment chambers and said divider walls are perforated.

13. The currency storage, purification and distribution system of claim 10 wherein a quantity of coinage may be stored within said plurality divider walls.

14. The currency storage, purification and distribution system of claim 10 wherein said cash drawer further comprises a front panel.

15. The currency storage, purification and distribution system of claim 14 wherein said front panel is perforated and further comprises a UV strip.

16. The currency storage, purification and distribution system of claim wherein said cash drawer further comprises at least one footer mechanism disposed at each lower side of the four corners of said cash drawer mechanism.
